(19) United States
(12) Patent Application Publication
(10) Pub. No.: US 2020/0390529 A1
(43) Pub. Date: Dec. 17, 2020

(54) STRUCTURE OF PROCESSING MATERIAL BLOCK OF COMPLETE DENTURE

(71) Applicants: WEN-HSIN CHIOU, Taichung City (TW); JHEN-GUO TSAI, Tainan City (TW)

(72) Inventors: WEN-HSIN CHIOU, Taichung City (TW); JHEN-GUO TSAI, Tainan City (TW)

(21) Appl. No.: 16/443,809

(22) Filed: Jun. 17, 2019

(51) Int. Cl. A61C 13/01; A61C 13/00

(52) U.S. Cl. CPC A61C 13/01; A61K 6/0023; A61L 2430/12; A61C 13/0004

(57) ABSTRACT

A processing material block of a complete denture includes; a disk body with a predetermined thickness; at least one positioning notch formed on a side of the disk body for installing/positioning a processing device; a horseshoe preform block disposed at an end apart from the disk body and having a height and first and second ends defined on opposite sides of the horseshoe preform block and being provided for the processing device to form a serrated arrangement structure; and plural tray preform blocks disposed at the first and second ends of the horseshoe preform block, each being higher than the horseshoe preform block. The structural type simulates the posterior margin of human teeth and is provided for the processing device to form the shape of the gum of the formed posterior margin of the teeth, and this structure can improve the stereoscopic and simulation levels of the finished product.

### FIELD OF THE INVENTION

The present invention relates to a processing material block, and more particularly to a novel processing material block structure used for manufacturing a complete denture and capable of providing a high level of simulation.

### BACKGROUND OF THE INVENTION

For the elderly and patients who have lost all of their teeth due to oral diseases, there is always a need for a complete denture or a denture restoration/reconstruction. Before the complete denture is made or a repair is designed and carried out, dynamic jaw relations must be recorded to ensure the applicability and comfortability of the subsequent products. Since the joint position of human real teeth and gum has a specific curve rather than a straight line, and the shape of upper and lower jaws varies from person to person, therefore a processing material block structure used for manufacturing a denture becomes very important. At present, most conventional processing material block structures used for manufacturing a denture are in a flat shape and have a straight intersection line to divide the denture into upper and lower different-color layers in order to show the color difference of the processed teeth and gum and compensate the color difference of the teeth and gum. Although such conventional processing material block is very popular and has been used for years, yet the intersection line of the teeth and the gum of the finished product cannot be the same as that of the real teeth, the level of simulation is low, and the appearance is poor. Manual coloring and repair are required additionally, and thus the convenience of processing is reduced significantly.

### SUMMARY OF THE INVENTION

In view of the aforementioned drawbacks of the conventional input/output network device, the inventor of the present invention based on years of experience in the related industry to conduct extensive research and experiment, and finally developed an improved structure of a processing material block of a complete denture in accordance with the present invention to overcome the drawbacks of the prior art.

Therefore, it is a primary objective of the present invention to provide a novel processing material block structure capable of improving the level of simulation, the aesthetic appearance, and the processing convenience of the complete denture.

To achieve the aforementioned objective, the present invention provides an improved structure of a processing material block of a complete denture, comprising: a disk body, in form of a disk with a predetermined thickness; at least one positioning notch, formed on a side of the disk body, for installing and positioning a processing device; a horseshoe preform block, disposed at an end apart from the disk body, and having a height, and a first end and a second end defined on two opposite sides of the horseshoe preform block respectively, and the horseshoe preform block being provided for the processing device to form a serrated arrangement structure; a plurality of tray preform blocks, disposed at the first and second ends of the horseshoe preform block, and each tray preform block being higher than the horseshoe preform block, and its structural type simulating the rear margin of human teeth and being provided for the processing device to form a gum shape of the rear margin of the teeth; wherein, after the processing device carries out a first processing means, the processing material block is unloaded into a material filling box, and a gum colored soft rubber material is injected, and after the gum colored soft rubber material is hardened, and after the processing material block is removed from material filling box, the processing material block shows a two-tone structural type.

The present invention will become clearer in light of the following detailed description of an illustrative embodiment of this invention described in connection with the drawings.

### BRIEF DESCRIPTION OF THE DRAWINGS

FIG. 1 is an exploded view of a processing block structure of a complete denture of the present invention;

FIG. 2 is a perspective view of a processing block structure of a complete denture of the present invention; and

FIG. 3 is a schematic view of an implementation status of the present invention.

### DESCRIPTION OF THE PREFERRED EMBODIMENTS

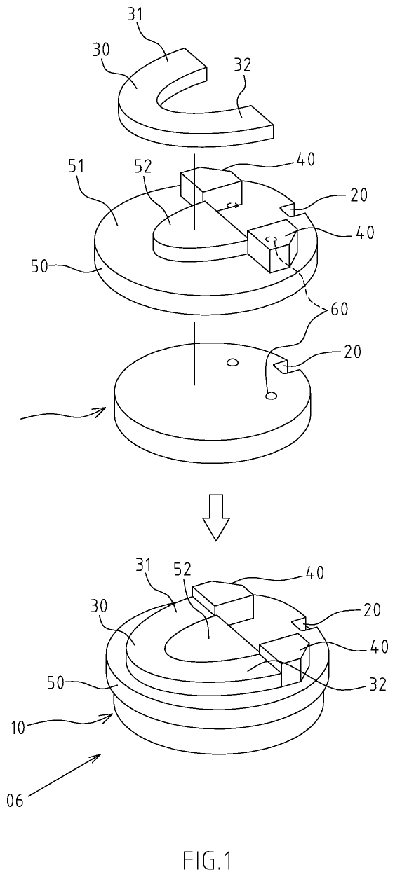

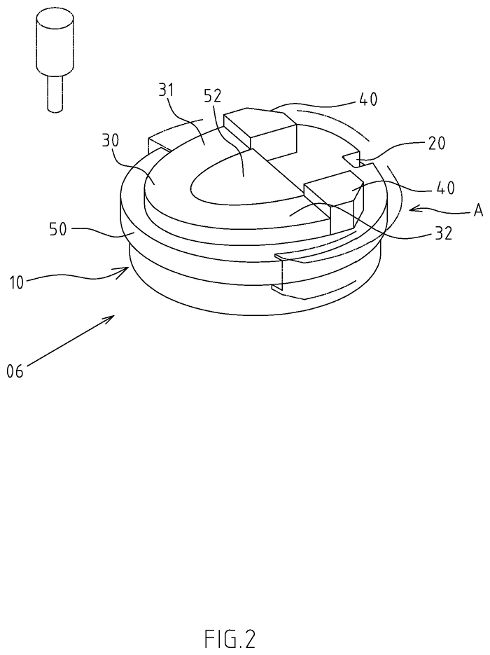

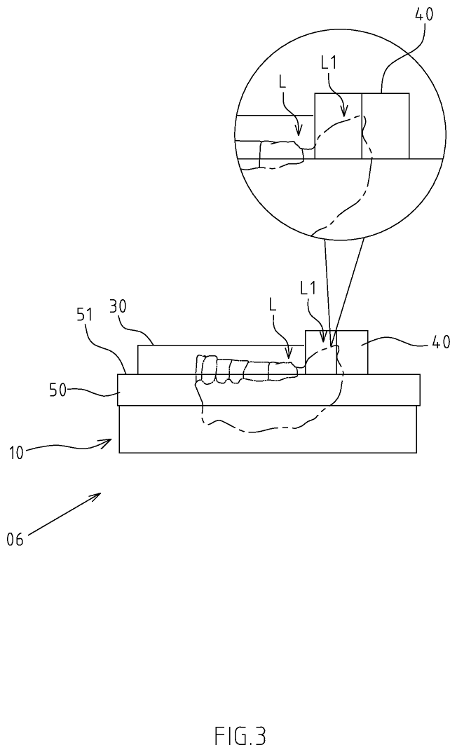

With reference to FIGS. 1 to 3 for an improved processing material block structure 06 of a complete denture in accordance with the present invention, the improved processing material block structure 06 comprises: a disk body 10, being a disk with a predetermined thickness; at least one positioning notch 20, formed on a side of the disk body 10, for installing and positioning a processing device (as shown in the label A of FIG. 3); a horseshoe preform block 30, disposed at an end away from the disk body 10 and having a height and a first end 31 and a second end 32 defined on two opposite sides of the horseshoe preform block 30 respectively, and the horseshoe preform block 30 being provided for the processing device to form a serrated arrangement structure (not shown in the figure); a plurality of tray preform blocks 40, disposed the first and second ends 31, 32 of the horseshoe preform block 30, and each tray preform block 40 being higher than the horseshoe preform block 30, and its structural type simulating the rear margin of human teeth and being provided for the processing device to form a gum shape (labeled as L in FIG. 3, and the labelled position is not formed yet, but it is just a previewed structure only) at the rear margin of the teeth (labeled by L in FIG. 3); wherein after the processing device carries out a first processing means (including but not limited to cutting), the processing material block 06 is unloaded into a material filling box, and then a gum colored soft rubber material is injected. After the gum colored soft rubber material is hardened and the processing material block 06 is removed from the material filling box, the processing material block 06 is in a two-tone structural type. It is noteworthy that the steps of injecting the gum colored soft rubber material into the material filling box, and removing the processing material block 06 are not shown in the figure.

Wherein, the tray preform block 40 is disposed at a position with an interval apart from an end surface 51 of a base 50, and an alignment flange 52 is protruded from the tray preform block 40 and provided for the horseshoe preform block 30 to lean on, so that the tray preform block 40 is disposed at the first and second ends 31, 32 of the horseshoe preform block 30. In an embodiment of the present invention, a positioning notch 20 is formed on a side of the base 50, but the invention is not limited to such arrangement.

Wherein, the horseshoe preform block 30, the tray preform block 40 and the disk body 10 are combined into a stacked form, and the position where the base 50 and the disk body 10 are aligned with each other has at least one alignment structure 60, so that after the horseshoe preform block 30, the tray preform block 40, the disk body 10 and the base 50 are stacked with each other, the processing material block 06 is formed into an integral structural type by pressing injection.

Wherein, after the processing material block 06 is removed from the material filling box, a second processing means (such as a detailed cutting, etc.) is carried out to show a two-tone complete denture finished product type.

In summation of the description above, the processing material block 06 in accordance with the present invention overcomes the drawbacks of the prior art effectively and has the effect of improving the convenience of the processing and the simulation level and aesthetic appearance of the finished product. The processing material block 06 in accordance with the present invention complies with patent application requirements, and thus is duly file for patent application and is submitted to the Patent and Trademark Office for review and granting of the commensurate patent rights.

While the invention has been described by means of specific embodiments, numerous modifications and variations could be made thereto by those skilled in the art without departing from the scope and spirit of the invention set forth in the claims.

What is claimed is:

1. An improved structure of a processing material block of a complete denture, comprising:
a disk body, in form of a disk with a predetermined thickness;
at least one positioning notch, formed on a side of the disk body, for installing and positioning a processing device;
a horseshoe preform block, disposed at an end apart from the disk body, and having a height, and a first end and a second end defined on two opposite sides of the horseshoe preform block respectively, and the horseshoe preform block being provided for the processing device to form a serrated arrangement structure;
a plurality of tray preform blocks, disposed at the first and second ends of the horseshoe preform block, and each tray preform block being higher than the horseshoe preform block, and its structural type simulating the rear margin of human teeth and being provided for the processing device to form a gum shape of the rear margin of the teeth;
wherein, after the processing device carries out a first processing means, the processing material block is unloaded into a material filling box, and a gum colored soft rubber material is injected, and after the gum colored soft rubber material is hardened, and after the processing material block is removed from material filling box, the processing material block shows a two-tone structural type.

2. The improved structure of a processing material block of a complete denture according to claim 1, wherein the tray preform block is disposed is disposed at an end surface of a base with an interval apart, and an alignment flange is protruded from the tray preform block, and provided for the horseshoe preform block to lean on, so that the tray preform block is disposed at the first and second ends of the horseshoe preform block.

3. The improved structure of a processing material block of a complete denture according to claim 2, wherein the horseshoe preform block, the tray preform block and the disk body are combined into a stacked form, and at least one alignment structure is disposed at a position where the base and the disk body are aligned with each other, so that after the horseshoe preform block, the tray preform block, the disk body and the base are stacked with each other, an integral structural type is formed by pressing injection.

4. The improved structure of a processing material block of a complete denture according to claim 1, wherein after the processing material block is removed from the material filling box and a second processing means is carried out, the processing material block is of a two-tone complete denture finished product type.
